(19) United States
(12) Patent Application Publication
(10) Pub. No.: US 2020/0253654 A1
(43) Pub. Date: Aug. 13, 2020

(54) BONE AND JOINT STABILIZATION DEVICE FEATURES AND DELIVERY SYSTEMS

(71) Applicant: PANTHER ORTHOPEDICS, INC., Sunnyvale, CA (US)

(72) Inventors: Kathryn A. Stecco, San Jose, CA (US); John Kent Ellington, Charlotte, NC (US); Carlos Castro, San Jose, CA (US); Frank P. Becking, La Canada Flintridge, CA (US)

(21) Appl. No.: 16/728,851

(22) Filed: Dec. 27, 2019

Related U.S. Application Data
(60) Provisional application No. 62/788,388, filed on Jan. 4, 2019.
(60) Provisional application No. 62/788,377, filed on Jan. 4, 2019.
(60) Provisional application No. 62/788,343, filed on Jan. 4, 2019.

(51) Int. Cl. A61B 17/84; A61B 17/86

(52) U.S. Cl. CPC A61B 17/842; A61B 2017/00867; A61B 17/8605

(57) ABSTRACT

Components and associated methods of manufacture or assembly and/or use for bone and joint stabilization devices or systems are described. Details hereof focus on features of the subject anchoring heads, spring members and associated handling features, delivery devices and/or kitted systems.

### RELATED APPLICATIONS

This application claims the benefit of and priority to U.S. Provisional Patent Application Ser. No. 62/788,343, filed Jan. 4, 2019 and entitled, “DELIVERY SYSTEMS FOR BONE AND JOINT STABILIZATION DEVICES,” U.S. Provisional Patent Application Ser. No. 62/788,377 filed Jan. 4, 2019 and entitled, “SPRING MEMBER FEATURES OF BONE AND JOINT STABILIZATION DEVICES,” and U.S. Provisional Patent Application Ser. No. 62/788,388 filed Jan. 4, 2019 and entitled, “ANCHOR RELATED FEATURES OF BONE AND JOINT STABILIZATION DEVICES,” all of which are incorporated by reference herein in their entireties for any and all purposes.

### FIELD

The embodiments described herein are related in the field of surgery and, more particularly, for use in bone fusion, joint stabilization and/or fracture fixation surgery.

### BACKGROUND

Various devices have been employed in orthopedic surgery for bone fusion and/or joint stabilization. Bone screws, staples and plates have served as a set of rigid options. Per U.S. Pat. Nos. 4,959,064; 6,656,184; 7,833,256; 7,985,222; 8,048,134; 8,449,574 and 8,491,583 and U.S. Publ. No. 2006/0264954 some screw-type devices have incorporated tensioning springs or members. Button-and-suture type devices have provided a more flexible set of options. U.S. Pat. Nos. 7,235,091; 7,875,057 and 8,348,960 offer examples of such device and suitable applications therefor. The subject embodiments address many shortcomings of existing products as may be appreciated by those with skill in the art in review of the present disclosure.

### SUMMARY

Bone and/or joint stabilization devices are described that are advantageously tensioned during a medical procedure to remain active in maintaining compression of associated anatomy during use. In various embodiments, an orthopedic surgery device or system comprises an elongate member or body, optionally comprising a spring pattern defined by a plurality of beams, each including a lateral component free to deflect when stretching the elongate body axially. An anchoring head typically receives the elongate body and may secure it with a one-way (e.g., ratcheting) interface. Two such anchors may be used, or one such anchor may be used at a proximal location with a deployable foot or a screw anchor used to anchor an opposite, distal end of the elongate body as described herein or as in U.S. patent application Ser. No. 16/032,736 and PCT/US18/41620 that are incorporated herein by reference in their entities for all purposes. Other details of the elongate spring member and anchoring head and foot features may be appreciated by reference to U.S. Publ. No. 2016/0213368 (now U.S. Pat. No. 10,194,946) and Int'l Publ. No. WO 2016/122944, both of which are incorporated by reference herein in their entities and for any and all purposes. Associated methods of medical use applicable to the subject devices or systems are presented in FIGS. 8-15 of the above-referenced publications.

Details of various embodiments are presented below. The subject device or systems, kits in which they are included (with or without assembly), methods of use (e.g., with implantation, during treatment of a patient while mending and/or for system removal) and manufacture (including assembly of the various components—as applicable—by a technician prior to sale or during a medical procedure by a surgeon) are all included within the scope of the present disclosure.

### BRIEF DESCRIPTION OF THE DRAWINGS

The details of the subject matter set forth herein, both as to its structure and operation, may be apparent by study of the accompanying figures, in which like reference numerals may refer to like parts. The components in the figures are not necessarily to scale, emphasis instead being placed upon illustrating the principles of the subject matter. The illustrations are intended to convey concepts, where relative sizes, shapes and other detailed attributes may either be illustrated schematically rather or precisely. To-scale features (e.g., as from engineering drawings and/or photographs) may be relied upon as antecedent basis for claim support.

FIGS. 1A and 1B are side-perspective views of different embodiments of the subject orthopedic implants.

FIGS. 2A and 2B are perspective detail and cross-sectional views, respectively, of an embodiment of an elongate spring member and an anchoring head configuration of the subject implants.

FIG. 3 is a face or top view of another embodiment of an elongate spring member section or pattern.

FIGS. 4A-4C are top, bottom and side-sectional views, respectively, of a first anchoring head embodiment incorporating Nitinol teeth. FIGS. 5A-5C are top, bottom and side-sectional views, respectively, of a second anchoring head embodiment incorporating Nitinol teeth.

FIG. 6 is a side view of another orthopedic implant embodiment, shown using anchoring heads as illustrated in FIGS. 4A-4C.

FIGS. 7A and 7B are front and rear isomeric views, respectively, of an embodiment of an anchor-handling or loading device. FIGS. 8A and 8B are perspective views of the same handling device with an anchoring head being loaded and in a loaded position, respectively.

FIG. 9 is a perspective view of an embodiment of a bone-screw tipped implant. FIG. 10 is a side view of a section of the spring member element for the embodiment. FIG. 11 is a perspective view of a bone-screw element for the embodiment. FIGS. 12A and 12B are perspective and sectional views, respectively, of a threaded interface element for the embodiment.

FIG. 13 is a side view of an implant embodiment including a proximal handling section and an (optional) integral distal needle for use in accordance with the implant approach of FIG. 1A.

FIGS. 14A and 14B are side views of embodiments relating to that of FIG. 1B (i.e., including a stowable anchoring foot) with integral and tied-on needles, respectively.

FIG. 15 is a side view with a side-perspective detail of another integral needle embodiment, in this case with a needle section extending from its anchoring foot.

FIG. 16 is a perspective side view of a system embodiment in which a clip-on needle is attached to a spring member body. FIG. 17A is a perspective view of the attachment region of the system shown in FIG. 16; FIG. 17B is a cross-section view of the detail region shown in FIG. 17A.

FIG. 18 is a side partial-section view of a delivery system suitable for use with the implant of FIG. 1A (i.e., a system using two anchoring heads).

FIGS. 19A-19D illustrate deployment steps for a delivery system suitable for use with the system of FIG. 1B (i.e., a system including a distal anchoring foot).

FIG. 20 is a perspective view of a delivery system that operates according to FIGS. 19A-19D. FIGS. 21A-21D are perspective views of components of the delivery system of FIG. 20.

FIG. 22 is a perspective view of another embodiment of a delivery system operable according to FIGS. 19A-19D. FIG. 23A is a section view of a distal end of the device; FIG. 23B is a section view of the proximal handle interface portion of the device.

### DETAILED DESCRIPTION

Various example embodiments are shown in the figures and further described below. Reference is made to these examples in a non-limiting sense, as it should be noted that they are provided to illustrate more broadly applicable aspects of the devices, systems and/or methods. Various changes may be made to these embodiments and equivalents may be substituted without departing from the true spirit and scope of the various embodiments. In addition, many modifications may be made to adapt a particular situation, material, composition of matter, process, process act(s) or step(s) to the objective(s), spirit or scope of the present invention. All such modifications are intended to be within the scope of the claims that can be made herein.

Regarding materials, the spring members may be laser-cut in NiTi alloy that is superelastic at human body temperature (37° C.) or below and subsequently electropolished. Other material options for the spring member include β-titanium alloys, certain higher performance plastics including poly-ether-ether-ketone (PEEK) or other materials with at least relatively high reversible strain properties. The anchors (heads or feet) may be molded in PEEK or machined in stainless steel or another material. Molded anchors optionally include markers or may be loaded with barium sulfate for radiopacity. Markers may take the form of discs or “pucks” pressed into pockets or may be in the form of a disc or rim attached to the marker. In the case of an anchor head, such a disc or rim is optionally round, in the case of an anchoring foot it may be oblong or racetrack shaped. Suitable marker materials include tantalum, stainless steel and even NiTi. Any cross pins used may be made of stainless steel, NiTi or another suitable metal alloy. The same is true of any screw heads, though they might alternatively be made of PEEK, especially if to be used in as a soft-tissue anchor. Many other material options exist and are not intended to limit the invention unless so-claimed.

The subject methods, including methods of use and/or manufacture, may be carried out in any order of the events which is logically possible, as well as any recited order of events. Medical methods may include any of a hospital staffs activities associated with device provision, implant introduction, positioning and/or re-positioning, and surgical access, closure and/or removal (e.g., as in an explant procedure).

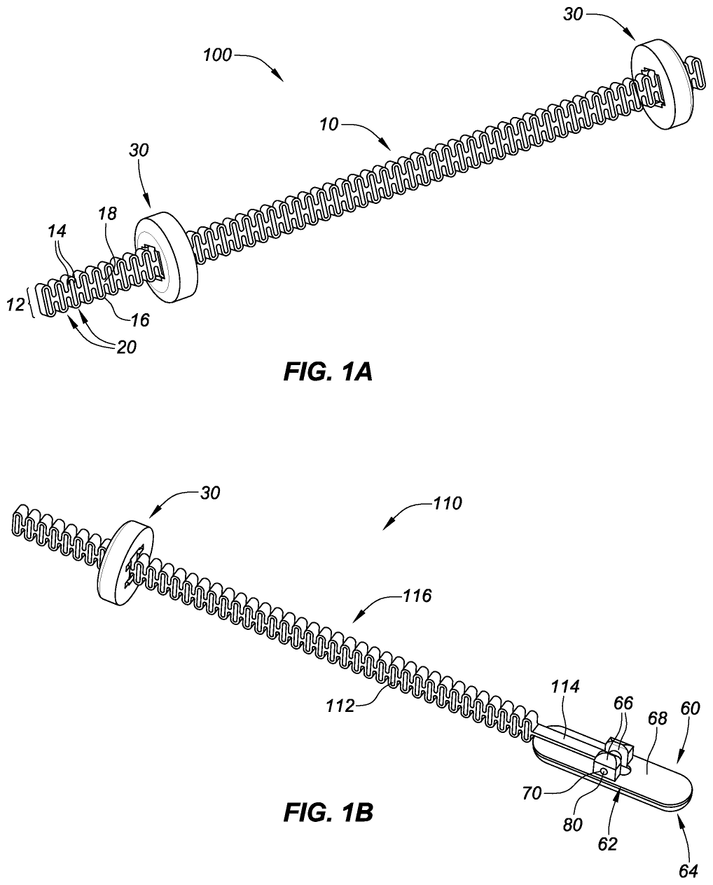

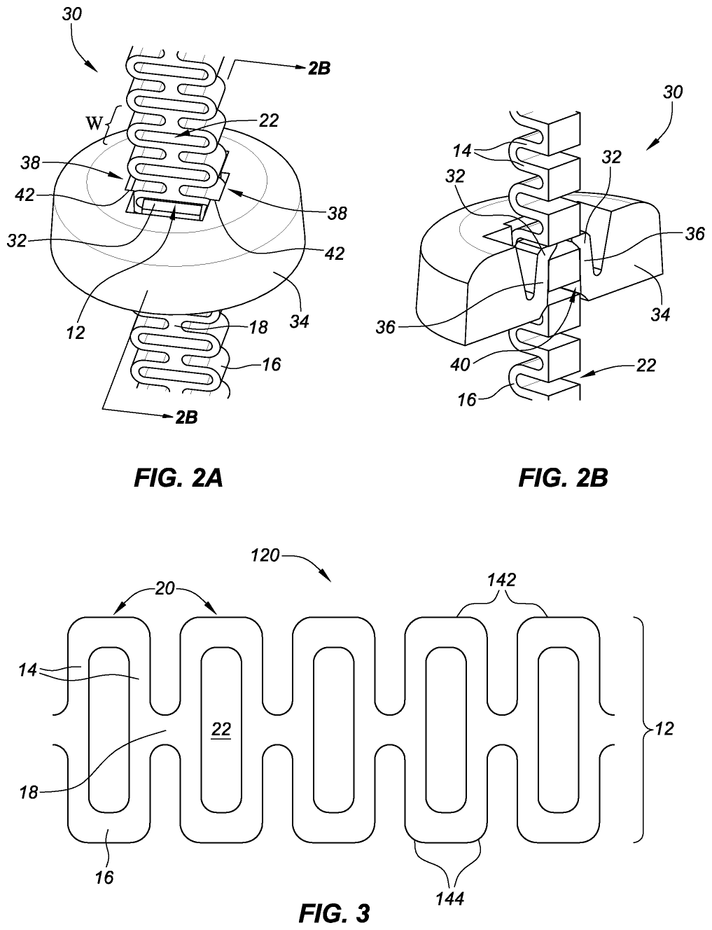

Embodiment 100 in FIG. 1A includes an elongate spring member or body 10 in the form of a stretchable or spring-type architecture including a plurality of beams 12, the beams each include a lateral component free to deflect for stretching the spring member axially. In the spring pattern, lateral bars 14 are provided in opposing pairs joined to each other at an outer extent connector 16 of each beam. Each such connector may be a curved continuation of each bar or beam member as shown in FIGS. 1A, 1B, 2A and 2B or otherwise configured (e.g., as shown in connection with FIG. 3 as squared-off—albeit radiused—ends). Each pair of opposing beams is connected to an axially adjacent pair by a medial connector or bridge 18. The beams or beam pairs serve as leaf spring elements in series that are arranged in cells 20.

Embodiment 110 in FIG. 1B includes a similar spring member section 112. The embodiment also includes a longitudinal extension section 114. Together, the spring and the axial or longitudinal extension sections define an overall elongate body 116. Embodiment 110 employs different anchoring features than embodiment 100. In embodiment 100, two opposite-facing one-way anchor heads 30 are used. In embodiment 110 only one anchor or anchoring head 30 is used together with a pivoting foot anchor 60. Either embodiment may be covered by a sheath prior to deployment. If implanted, the sheath may prevent tissue ingrowth.

In embodiment 110, a socket with a through hole or aperture (not shown) is formed at the end of extension 114. The anchor or anchoring foot 60 in embodiment 110 may comprise a body 62 with an oval, race-track or rectangular planform shape. Generally, the height, length and width of the foot will be minimized while still maintaining adequate surface area and strength for load bearing. The distal or outboard surface 64 of the foot may be fully radiused to decrease crossing profile and/or to improve or enhance the interface with overlying tissue without significant loss of strength. Bosses 66 extend above a proximal or inboard surface 68 of foot 60. A transverse hole 70 is formed in each boss. A pin 80 is received through each of through holes 70 and the extension 114 aperture to attach anchoring foot 60 in embodiment 110. So-connected or affixed, the anchoring foot can rotate from a position aligned with the elongate body to a position transverse (or at least angled, typically upwards of about 45 or about 60 degrees up to 90 degrees) to the elongate body for anchoring the overall device during a medical procedure.

In FIGS. 2A and 2B, detailed aspects of the anchoring head 30 in FIGS. 1A and 1B are shown. The anchor or anchoring head may be designed for one-way advancement over the spring member body 10 or body section 112 as stated above. As shown, at least one tooth 32 in each anchoring head interacts with the apertures or windows 22 defined within each cell 20 of the spring body or portion.

The overall shape of the anchor head body 34 may be round, square or otherwise configured. Indeed, the support structure (i.e., the body) for included support columns 36 and teeth 32 in a given anchor head may be integrated in an orthopedic plate (e.g., as integrally formed or press-fit therein) or otherwise provided.

Guide slots 38 for the spring member body 10 or section 112 may be provided in the anchoring head 30 to ensure even engagement with teeth 32. To further stabilize the spring member body or section, the support columns 36 may be configured with an inner surface 40 that parallel the side faces 42 of the slot as much as possible (i.e., given molding draft angle considerations). Further, the spring member may be configured to coordinate further with the guides 38. For example, a spring member pattern 50 may include flattened sides 52 as shown in FIG. 3. To produce these shapes, the external radii 54 of connections between adjacent beam pairs at their lateral extent may be minimized and/or the lateral connectors 16 between adjacent sets of beams lengthened. These (relatively extended) flat section(s) 52 provide further means of ensuring spring member guide slot retention.

However, these features are optional as round-ended elongate spring member cells such as shown in the preceding figures and the disclosures incorporated-by-reference herein are well-retained within the guide features. New features for use in connection with and/or replacing the features described above are presented below.

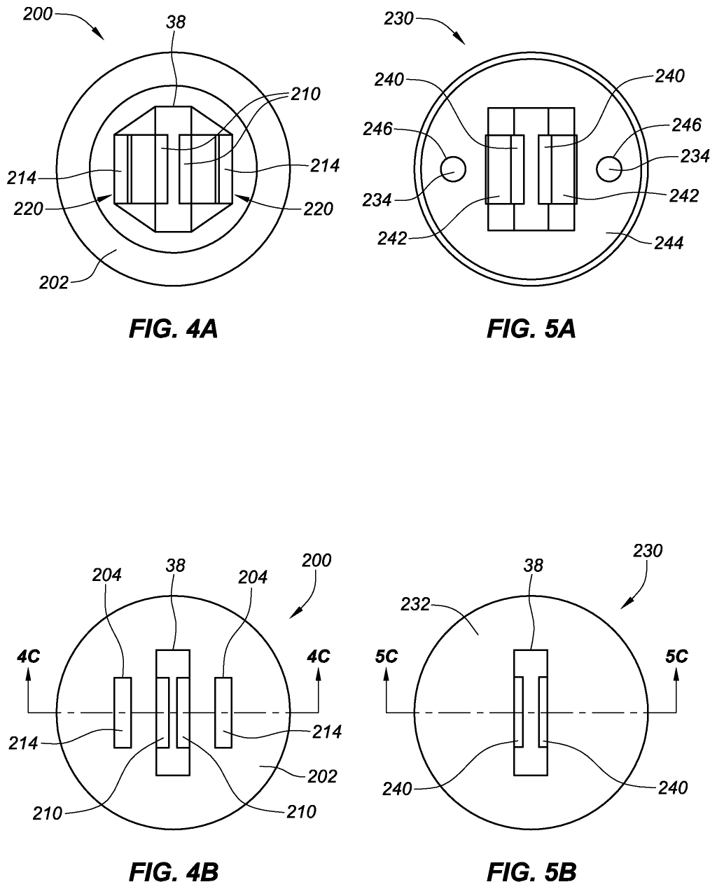

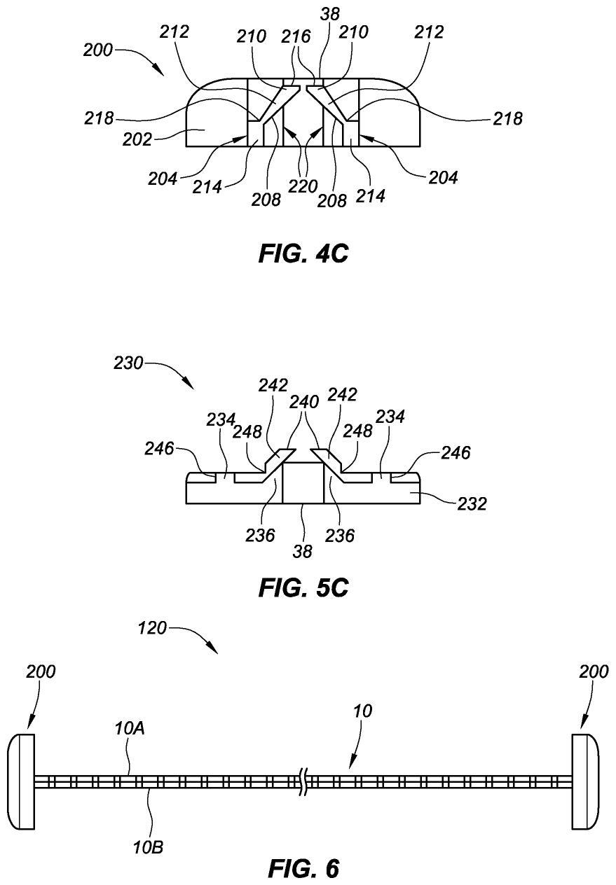

In one example, FIGS. 4A-4C show top, bottom and side-sectional views, respectively, of an anchoring head embodiment 200 incorporating Nitinol teeth and associated features pressed into an (optionally plastic) anchor body 202. Teeth and their associated supports portions (optionally referred to as columns) are produced in superelastic NiTi alloy (i.e., Nitinol) in these anchoring heads 200. Together, each tooth 210 to interface with the spring member body (e.g., 10 or 110, above), its support column 212 and a boss 214 may be regarded as an anchor retention body 220. Each anchoring head may include two such bodies 220 as shown, together with a guiding groove 38 and other features as described above for FIGS. 2A and 2B in a more general sense. Each retention body base or boss 214 is optionally configured for a press fit within a pocket or socket 204 of the anchor body 202.

Each tooth is shown including a flat landing or plateau 216 that interfaces with the interior surface of beams 12 of an implant spring member (or other member engaged therewith). However, this interface between the members may be otherwise configured.

Constructed of metal, the tooth is able to maintain integrity up to higher forces than a tooth of comparable geometry made from plastic. Nevertheless, actuation or insertion force (i.e., for moving the tooth up-and-out for clearance during spring member advancement) may be reduced by using a support column that tapers between its tooth and body boss (i.e., by producing an architecture that possesses a pivot or living hinge section 218). Even constructed of Nitinol, actuation or insertion can be improved relative to an anchor altogether made of PEEK.

Relative overall strength is improved not only by material selection. Each support arm or column 212 of each anchor retention body 220 may be backed by a body support section 208 as shown in cross-section per FIG. 4C. This section is angled (e.g., between 30 and 60 degrees or at an angel of about 45 degrees to a planar base or flat underside of body 202 of the anchor body as shown) and backs-up or supports the retention body column 212 when the spring member is under tension, pulling into the support surface.

Yet, the support section does not constrain support column flex away from the surface. The configuration permits separation or flex away from the support surface when loading or advancing the spring member through the anchoring head.

At the reduced junction 218 between the support column or arm 212 and the boss 214 in configuration shown in FIG. 4B, the NiTi alloy from which the retention body is made is able to deform significantly more without plastic deformation (by production of stress-induced martensite). As such, greater back-and-forth movement of the tooth surface is permitted (again as compared to an all-polymer tooth-and-column approach). The additional range (offered without a loading-force penalty, or even providing improvement) allows for greater depth of tooth insertion into a spring member body 10 or section 112.

This approach offers potential for a more robust support interface, reducing stresses on each of the tooth and retained section(s) of the spring member. In addition, using metal teeth (as executed in NiTi alloy, another titanium alloy such as β-titanium or another material) as described enables coordinated use with relatively narrower spring member bodies and associated (also more narrow) teeth without loss of strength relative to a wider or otherwise larger polymer (e.g., PEEK) tooth design.

For manufacture, such teeth (and associated sections of a retention body) are optionally laser (e.g., by fiber laser or femtolaser), water jet or wire electrical discharge machining (EDM) cut. They may be electropolished. So constructed, they can be produced to tight tolerance suitable for easily press-fitting with complimentary-shaped PEEK (or another polymer) anchor bodies 202. Alternatively, the retention bodies 220 may be overmolded with the PEEK in a single assembly.

FIGS. 5A-5C are top, bottom and side-sectional views, respectively, of a second anchor embodiment 230 incorporating NiTi alloy teeth 240 and associated features formed in connection with a cap or cover plate 244 to an anchor body 232. Similar to the previous embodiment, each support column or arm 242 may end at or include a reduced thickness pivot or hinge section 248. In the case of this anchoring head embodiment 230, however, the (living) hinge serves as a junction to a common base (i.e., cover plate 244).

Also distinct, instead of tapering the support column(s) as shown in embodiment 200, this junction between the cover and the support columns 242 may be relieved or notched to form the hinge section 248. In a (optionally) disc-shaped member with integrally formed (e.g., by laser cutting) teeth as shown, the relief may be formed by a grinding procedure after teeth and support columns are originally cut in a flat pattern are then heatset into the configuration shown. (The heatsetting or shape setting may be accomplished by exposing the Nitinol piece to between 500 and 550° C. for between about 5 and 15 minutes in a furnace or for a shorter time in a molten salt pot bath.) The teeth 210 may also include a flat 246 formed using a grinding procedure.

The cover or retainer plate 244 (along with teeth 240 and their support columns 242) may be secured to a polymer anchor body 232 via press-fit with bosses or pegs 234 formed in the body that are received by through-holes 246 of the base. Alternatively, a slip fit between the elements may be secured by heat-staking the plastic within the holes in the metal.

As in embodiment 220, the tooth support columns or beam 242 are backed by angled body sections 236 to prevent downward (backward relative to the spring member advancement) flex. A cap (not shown) to the embodiment in FIGS. 5A-5C can be added to match the dome-shape profile of the FIG. 4A-4C embodiment as well.

FIG. 6 is a side view of orthopedic implant embodiment 120, optionally using anchoring heads 200 as shown in FIGS. 4A-4C. As referenced above, such embodiments used in connection with the anchors may be generally narrower, along with the teeth (and corresponding support columns, etc.) in the anchoring head than devices using the anchor 30 detailed in FIGS. 2A and 2B. However, an implant using a spring member 10 comprising two spring member layers 10A and 10B as shown in FIG. 6 may be also be constructed or provided in connection with such anchors 30. The same holds true with respect to using an anchoring foot 60 in connection with body layer(s) 112 and any of the anchoring head embodiments 30, 200 or 230.

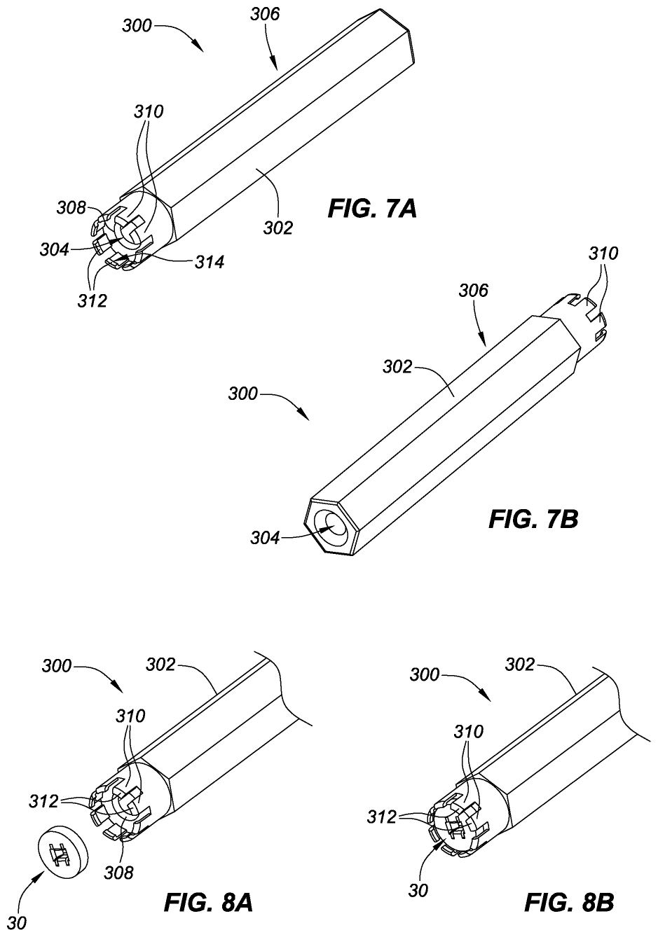

FIGS. 7A and 7B illustrate another plunger-type anchor loading device 300 suitable for used with any such anchoring head. Loader 300 has a body 302 that includes a through-hole or channel 304 to allow passage of a spring member body 10 or 110. It also includes a plurality flexible extensions or “fingers” 310 with overhanging catch portions or tips 312. An undercut ramp section 314 of each tip to allow anchor release when desired. The fingers are narrow and thin enough to allow the necessary flex to accommodate such action. Eight independent fingers are shown, but as few as three (typically symmetrically disposed) may advantageously be employed. The proximal “handle” portion 306 of the loader may be hexagonal as shown. Alternatively, it may be round. It may be between about 1 and 2 inches in length. Likewise, it may be between 0.25 and 0.75 inches in diameter. It may be injection molded or machined for manufacture and include a textured surface or additional features for user grip where handled.

FIG. 8A illustrates loading an anchoring head 30 in the plunger 300 or (alternatively) the reversable release therefrom. The domed geometry of the anchoring head 30 and (optional) mating socket feature 308 of the loader prevents units from being installed or assembled incorrectly (i.e., backwards). Correctly seating (and releasing) an anchor from the fingers may also provide a tactile and/or audible feedback (e.g., click).

Anchor loading into the plunger may be done manually by a user or it may be done in advance such that the anchor and loader or plunger are provided in “kit” fashion. Multiple preloaded anchor/plunger devices may be provided in packaged combination with a spring member as a system provided to physicians.

In any case, FIG. 8B shows the anchor loaded into and held by the plunger 308 until intended release. Such release is accomplished after a user advances the anchoring head to the desired position along the spring member body 10 or portion 112. Then, the user simply pulls with enough force to allow loader fingers 310 to flex and release their grip (from overhanging tips 312) on the anchor.

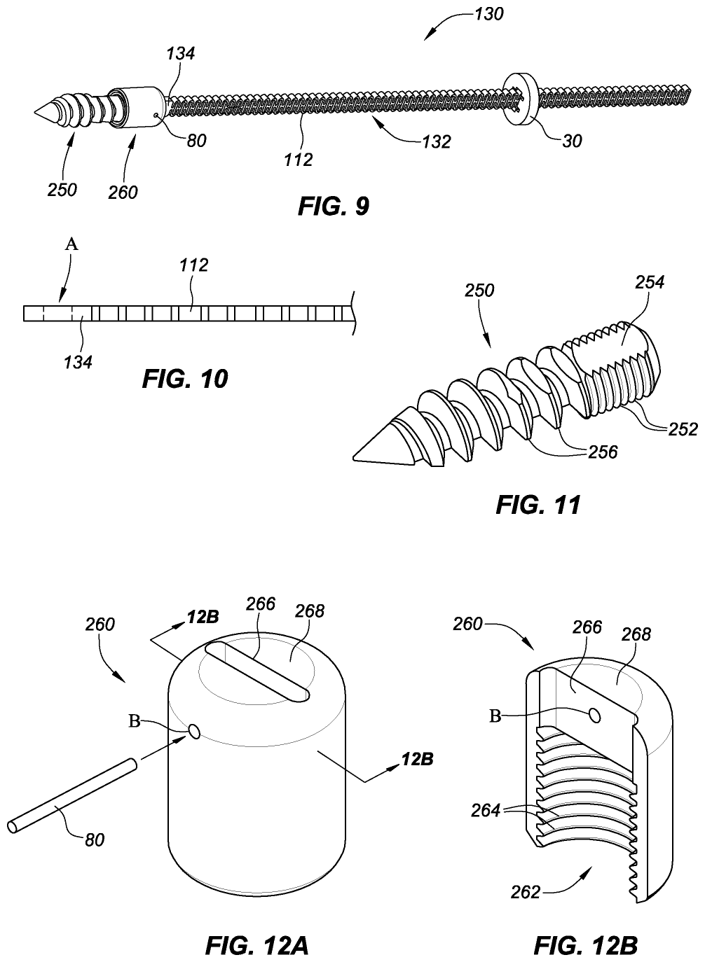

In another aspect hereof, FIG. 9 provides a perspective view of a bone-screw tipped implant embodiment 130. FIG. 10 is a partial side view of the spring member portion 112 as a component part. FIG. 11 is a perspective view of the subject bone-screw element or tip component 250. FIGS. 12A and 12B are perspective and sectional views, respectively, of a interface element 260. It includes a distal socket 262 with machine threads 264 matching the machine threads 252 of a proximal side of the bone-screw tip 250. A proximal slot in the socket 266 is configured to receive a rectangular tab 134 (in a manner similar to direct receipt in the screw head described in FIGS. 10A and 10B embodiment in U.S. patent application Ser. No. 16/032,736 filed Jul. 11, 2018 incorporated herein by reference in its entirety any further associated description) with a pin 80 pressed-fit through an aperture “A” at the end of an implant end aperture(s) “B” in interface element 250.

In use, the bone-screw element 250 is driven with bone-engaging coarse threads 256 into place through a bone tunnel with a trocar or similar instrument interfacing with one or more flats 254 across machine screw section 252. For such purpose, the drive may have a D-shaped or Double-D shaped recess or socket. Next, the machine-threaded socket interface element 260 (together with the implant body) is connected (i.e., screwed on to) to the bone-screw tip 250. It may be driven by a trocar or similar instrument interfacing with a tab section 134 of the implant body 132 extending proximally to interface element 262 as shown in FIG. 9 or otherwise. The manner in which tab 134 extends above the face or shoulder 268 of interface element or socket 260 provides drive surface(s) extending at least about 1 mm for interface with a complimentary driver tool (not shown).

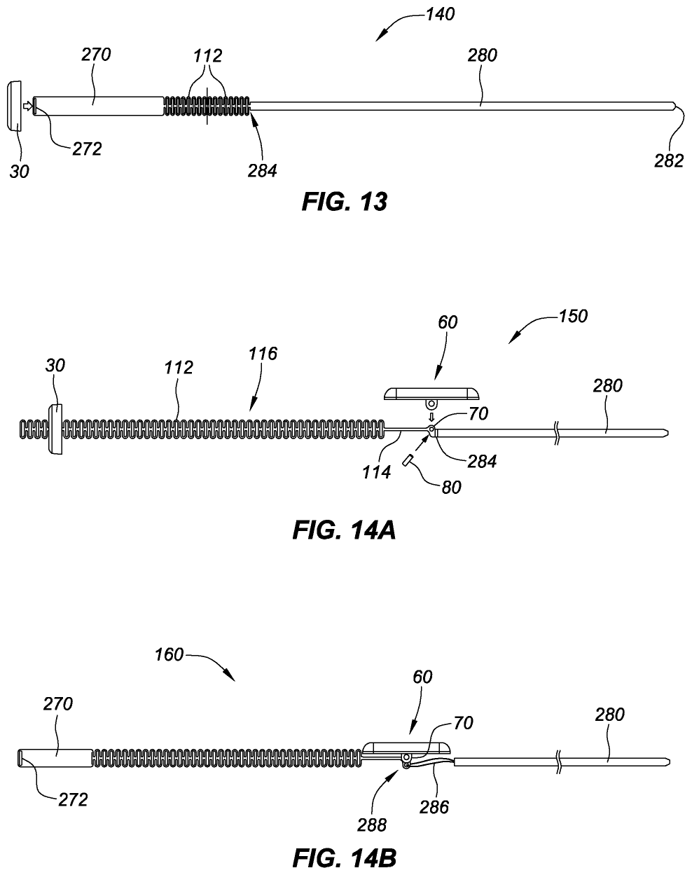

In another aspect hereof, FIG. 13 is a side view of an implant embodiment 140 including a proximal handling section 270 and an (optional) integral distal needle 280 for use in accordance with the dual anchoring head approach illustrated in FIG. 1A. The proximal handling section or tab 270 is advantageously sized to receive and hold (via an included slot or window 272) a pre-installed anchoring head. As such, the handling section (optionally, a “handle”) may be approximately as wide as the spring body portion 112 of the device.

Alternatively, the handling section may be wider and not fit an anchoring head. Optional needle section 280 may be advantageously narrower than the spring member body section 112. It may have a pointed tip 282 as shown to function as a true “needle” or the tip may be rounded/atraumatic in configuration (and yet still be referred to as a needle section).

Either way, the length of the needle section may be between about 100 and about 150 mm in length or longer. The spring member body section 112 may be between about 60 and about 100 mm in length. The proximal handling tab 260 may be between about 20 mm and about 60 mm, or about 40 mm in length. It may be between about 2 mm and 3 mm in width. All of these elements may be integrally formed as cut (typically laser cut, followed by electropolishing) in plate or ribbon (optionally superelastic NiTi material) that is between about 0.5 and about 1.5 mm thick, optionally about 1 mm thick or otherwise. In which case (i.e., when produced by laser cutting 1 mm thick plate), the needle section may have a substantially square cross-section if cut to 1 mm width (or stated otherwise, diameter).

In use, after threading the implant through a clearance hole or tunnel made in one or more bones, needle section 280 is trimmed off at the reduced-width “waist” or notched section 284 provided and an anchoring head 30 (or 200 or 230) loaded onto the spring member or body portion 112. If an anchoring head is preloaded as indicated on the proximal side of the device and held at the included window or aperture 272 (shown located adjacent the proximal end of the handling tab, but optionally placed elsewhere), the anchoring head 30 will be advanced onto the spring member section 112 before either handling tab 270 is trimmed off, or the spring member body section 112 is simply trimmed to length with an anchor head installed on the other end of the device.

The anchoring head for the distal side of the device can be similarly advanced along the length of needle section 280 and onto the body before trimming. If the system is to be used in this fashion, the notch or waist may be omitted (as the spring member body itself may be trimmed) and it may be advantageous to make the needle section wider—even up to the width of the body (just as the proximal tab section). If the needle is to be used for anchor loading as such, the needle may be tapered on its top and bottom surfaces instead of being tapered on its sides (as shown).

Whereas embodiment 140 shown in FIG. 13 relates to an implant approach of FIG. 1A, the embodiments shown FIGS. 14A and 14B relate to the approach FIG. 1B. Device embodiment 150 in FIG. 14A includes an integral needle section 280 added past its extension 114. As shown, an “upper” (relative to the drawing page) surface of the needle is aligned with that of the extension. This approach conserves space and minimizes crossing profile for the attached foot 60 by allowing it to lay flat across the surfaces. Overall, the needle may be sized as stated above. In any case, this embodiment represents one example in which the implant includes an oblong anchoring foot 60 that is rotatably connected at the end of an elongate spring member 116 (optionally to an extension 114 section thereof extending from a spring member section 112) and the introduction needle 280 extents past the oblong anchor.

In this particular embodiment, however, the included waist section 284 is advantageously oriented in a perpendicular or orthogonal direction to that of the spring member cut pattern. This allows for easy trimming with side cutters with the anchoring foot pinned in place (as shown). Post laser-cutting machining or secondary laser cutting (after re-orienting by turning the device 90 degrees) may be employed to produce the (optional) waist section 274 in this embodiment. It is also notable that with needle 270 in place, rotation of the foot 60 is prevented during implant advancement through and past a bone tunnel.

Device embodiment 160 in FIG. 14B employs a needle 280 secured by one or more fiber strands 286 (optionally comprising suture material) through an eyelet, particularly a secondary eyelet 288 formed in the implant body, at the end of extension 114 adjacent the anchoring foot pivot pin hole or eyelet 70 (which receives dowel or pin 80 to secure anchoring foot 60 to the implant body extension 114). Strand(s) 286 may be secured inside the body of the needle by swaging or other mean. In any case, needle 280 may be easily trimmed-off the remaining portion of the implant using scissors, a scalpel or another cutter.

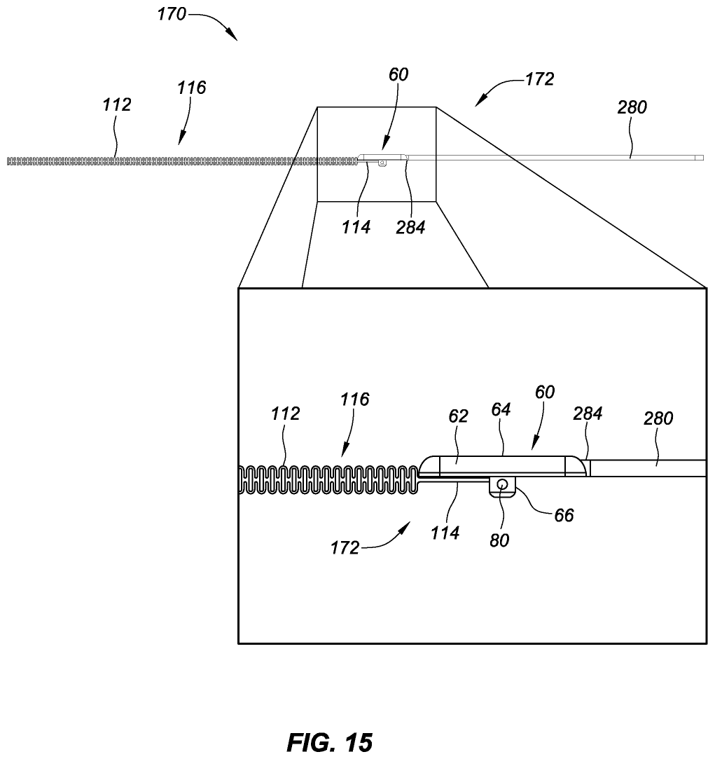

Device embodiment 170 shown in FIG. 15 resembles that in FIG. 14A with the exception that the integral needle section 280 extends from anchoring foot 60 of the device. Notwithstanding that difference, the construction still includes a waist or notch section 284 to aid with and define a cut-off location between the associated parts (in this case between the anchoring foot and the needle). The needle can be sharpened or angled on 2 sides as shown, on 3 or 4 sides or be conical in shape (just as the other needles above). The anchoringfoot-plus-needle part 172 can be machined or produced using plastic injection molding or metal injection molding (MIM). The needle may be square (as shown) or round in cross section as facilitated by the selected manufacturing technique. In any case, the construction may simplify manufacture by laser-cutting the body section alone.

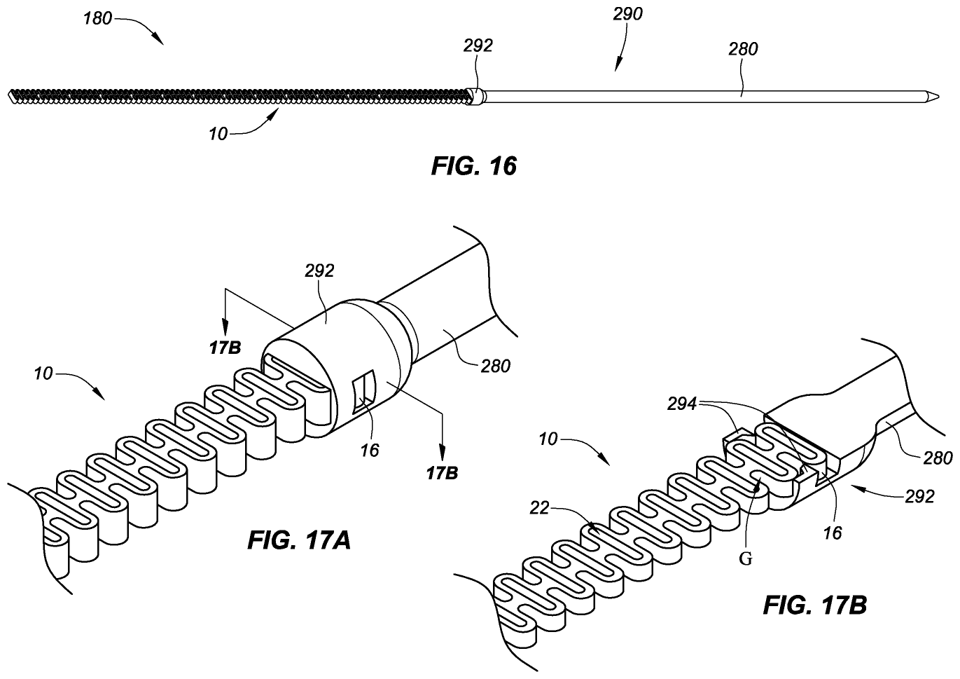

Like embodiment 150, embodiment 170 may offer certain advantages as the integral needle will stabilize anchoring foot position for delivery without need for a sheath or other similar means. In any case, each of the embodiments in FIGS. 14A, 14B and 15 offers its own distinct advantages. Also, the included needle 280 (in any such case) may be sized as described in connection with FIG. 13, above, or otherwise. The same is true for the embodiment shown in FIG. 16.

In FIG. 16, a system embodiment 180 includes a clip-on needle interface 290 attached to a spring member body 10. Needle interface 290 may be machined or injection molded plastic (e.g., PEEK). As such, it may include a needle section 280 and flexible features that can hold on to and then release an inserted spring member body. These features are formed in collar 292 shown in FIG. 17A. As shown in FIG. 17B in cross-section, teeth 294 may be included that releasably engage in gaps “G” between the sides 16 of the spring member 10. Alternatively, the teeth may be formed to fit within the windows or apertures of the spring member. In any case, these teeth may alternatively be referred to as detent features.

In the configuration shown with curved end connections 16 between beams 12 in the spring member 10, the needle's flat-top teeth 294 will be able to disengage in a system able to release the needle when pulled with at least about 1 or 2 pound of force (lbf) and typically less than about 5 lbf. Alternatively, the “teeth” may be rounded or ramped in both (top and bottom or proximal and distal) directions. Such features may advantageously be used in the case where the system is configured to retain the spring member via its window apertures 22 that (themselves) lack significant rounding.

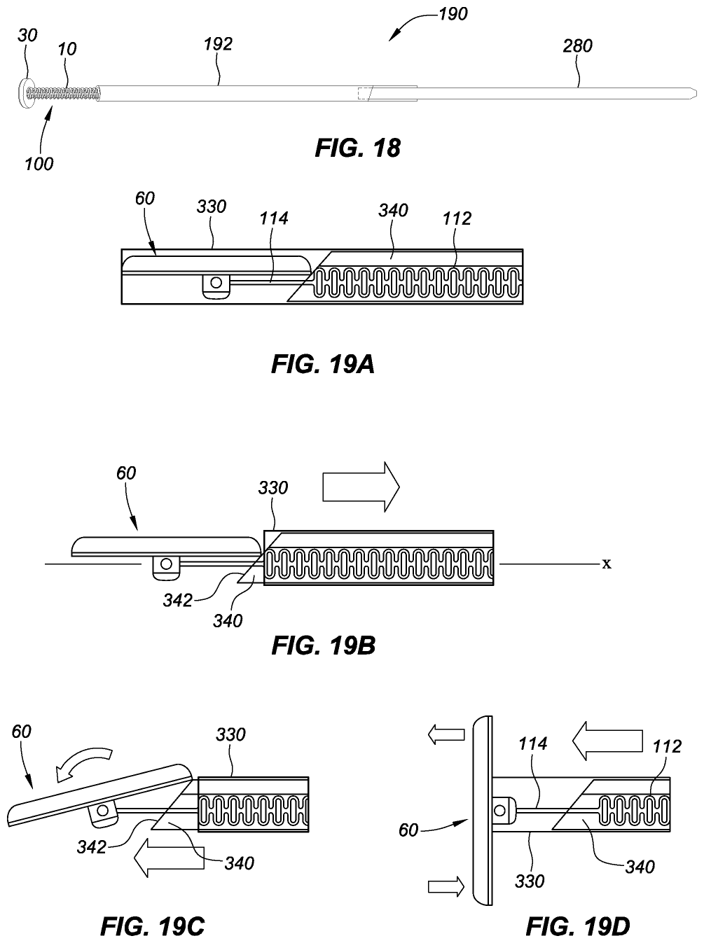

FIG. 18 illustrates another delivery needle approach for an implant 100 as shown is shown in FIG. 1A. The delivery system 190 includes a tunnel or tube 192 in the form of a metal (e.g., stainless steel) hypotube or plastic tubing tipped with an integrated advancement needle 280. The needle is shown connected to the hypotube (in partial cross-section) via a press fit. Other options are possible as well. In any case, tube 192 is sized to receive the body 10 of an implant 100.

The length of the needle section (extending beyond the tube into which it is press-fit or otherwise secured) may be between about 100 mm and about 150 mm or more. The open section of tube 192 may be long enough to receive all or substantially all of the implant body 10. In FIG. 18, the body is capped with an anchor 30 that serves as a limiter or stop for advancement within the tube. The needle may be metal (e.g., stainless steel) or plastic (e.g., PEEK or nylon such as PEBAX). It may be pointed or terminate with an atraumatic tip (as shown).

FIGS. 19A-19C illustrate deployment features and steps for a delivery system approach suitable for use with implant 110 of FIG. 1B. In FIG. 19A, anchoring foot 60 is coved by an outer sheath 330. This facilitates advancement through a drill hole “tunnel” created across the anatomy to be treated. In FIG. 19B, the anchoring foot is exposed. This may be accomplished by withdrawing sheath 330 (as indicated by the arrow) or advancing the anchoring foot in relative fashion. In FIG. 19C, a pusher 340 is advanced (or the foot withdrawn) into contact with each other. An angled face 342 of the pusher (e.g., with its angle set between about 20 and about 60 degrees relative to an axis “X” of the device) rotates the anchoring foot as indicated in FIG. 19C. Given its wedge-shaped face, it cleanly picks-up (vs. jams with) the proximal-facing end of the foot 60 and drives it to pivot outward (i.e., as pictured).

Then, the position of the anchoring foot is driven to its extent of rotation as shown in FIG. 19D. This may be accomplished by driving the sheath 330 forward (as indicated by the arrow) or withdrawing the anchoring foot into contact with the sheath. Once so-positioned, the sheath and pusher are withdrawn (not shown).

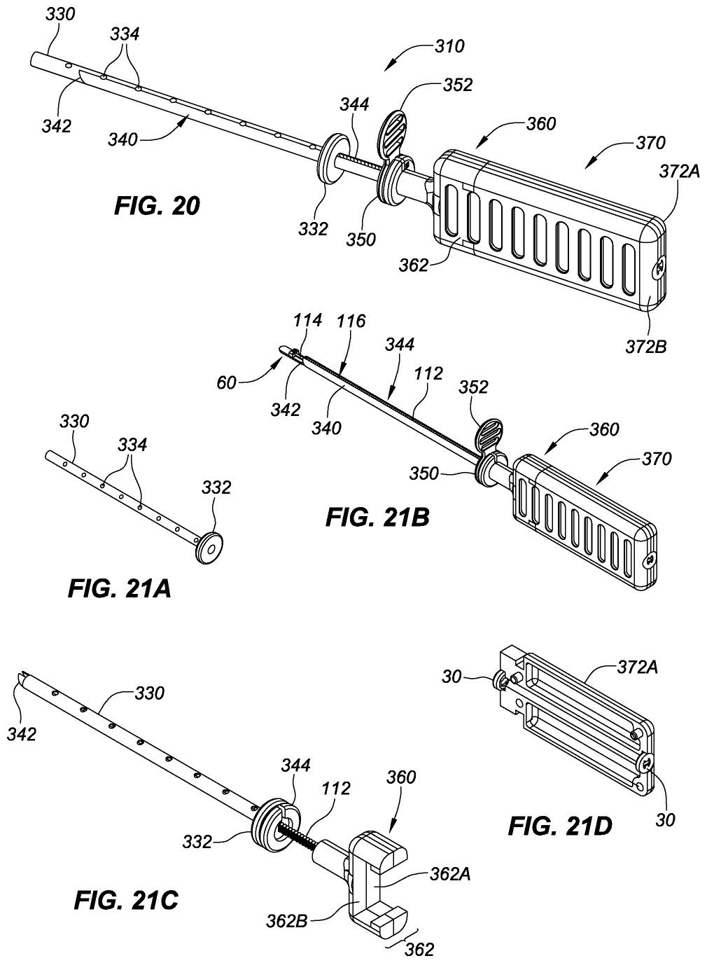

Pusher 340 may be a slotted body having an open channel 344 to receive an implant body 116 and extension section 114 as shown (in semi-transparent side view in FIGS. 19A-19D and variously in each of FIGS. 20, 21B, 22 and 23A and 23B) to offer a maximized face surface area for contact with anchoring foot 60 to manipulate the same, while maintaining a minimum diameter. Otherwise, the pusher may be a relatively thick-walled (e.g., 0.010 inch or more) tube (not shown) receiving the implant therein and use its angle-cut end for such contact.

FIG. 20 shows a manually-operated embodiment 310 to effect the action shown in FIGS. 19A-19D. The component parts include the sheath 330 also shown in FIGS. 21A and 21C. The sheath optionally includes a disc-shaped user interface portion or hub 332. It may also include reliefs/divots or through holes 334 to gauge length radiographically. The slotted pusher 340 shown in FIG. 21B is received within sheath 330 in FIG. 20. Its insertion depth (e.g., that of the pusher relative to the sheath) is limited by a removable collar 350 with a pull-tab interface 352 so that the sheath 330 will maintain the anchoring foot 60 of the implant 110 aligned with the spring member body 116 for advancement into place.

Once the tab is removed, however, pusher 340 and sheath 330 can assume an arrangement as shown in FIG. 21C (shown without the distal section of the implant) bringing hub 332 of the sheath into contact with a hub 344 of the pusher. With an implant in place, angled end 342 of the pusher can pivot the implant's anchoring foot 60 into position as described above. Also shown in FIG. 21C, the delivery system further includes a bracket or stirrup 360. This can be split open into two body pieces 262A and 262B to release the implant body 112 (an exaggerated length shown) from a form-fitting grip (such a feature is shown in FIG. 23D). Or a friction-type grip (not shown) may be employed within a single body 362 that need not be split to effect spring member body release.

In any case, bracket 360 also may releasably hold a handle or handling interface 370 for one or more anchoring heads. FIG. 21D shows a body portion 372A (an opposing body portion 372B is shown in FIG. 20) of the handle in a state of partial assembly (e.g., illustrating how it may be loaded with the anchoring heads 30 pictured.)

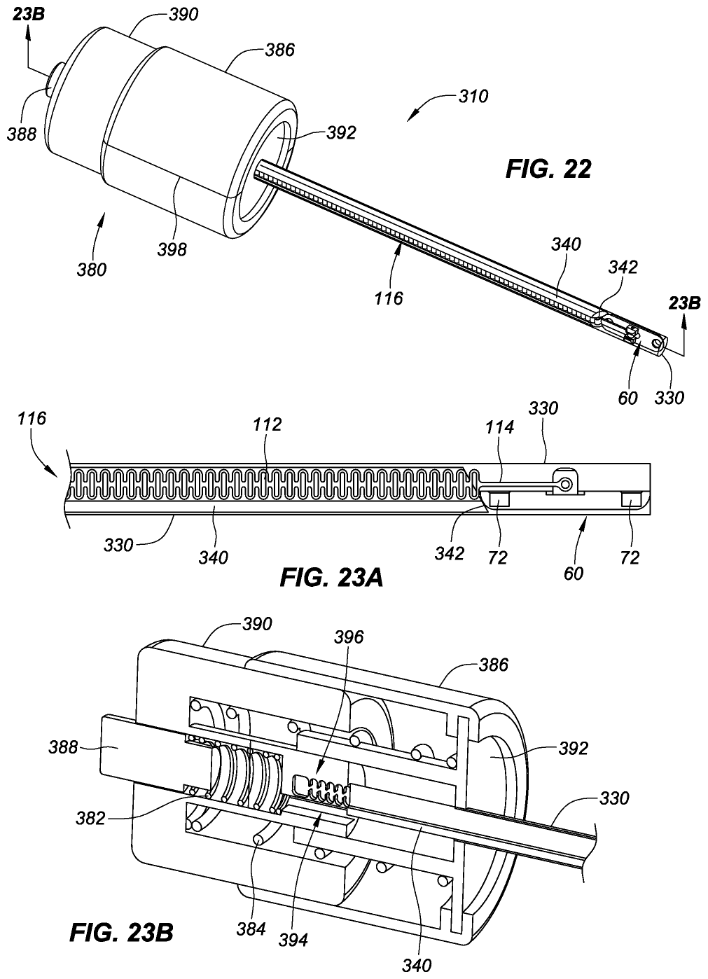

FIG. 22 is a perspective view of another delivery system embodiment 320 operable according to the approach shown in FIGS. 19A-19D. Like the system shown in FIGS. 21A-21D, it includes a sheath 330 and pusher 340. An implant body 116 with a rotatably attached anchoring foot 60 is loaded in the system as well. Associated details are pictured in the cross-sectional views of FIGS. 23A and 23B. For example, FIG. 23A clearly illustrates the inclusion of radiopaque markers 72 (as referenced above, these may comprise any radiopaque material commonly used such as tantalum or may even be NiTi plugs or pucks press-fit into plastic body 62 material) in the anchoring foot 60 of the implant along with the sheath 330 and pusher 340 components of the delivery system.

FIG. 23B illustrates constructional details of an actuatable handle assembly 380 of the delivery system (i.e., with the implant body removed from the assembly). The handle assembly includes a relatively smaller and higher spring rate inner spring 382 and a relatively larger and lower spring rate outer spring 384. So, with the outer cover 386 held stable when the core button 388 is depressed, the surrounding cap 390 is advanced along with the pusher 340 and the implant body 116, relative to the sheath attached to the cover 330 via flange section 392. This frees the implant's anchoring foot from sheath constraint.

Once cap 390 bottoms-out (e.g., within cover 386), core button 388 is advanced further thereby driving the associated pusher 340 forward, optionally via a pushrod (not shown) received within channel 394 of cap piece 390. Note that the pushrod may be an extension of either one of the core button 388 or pusher 340, it may be a discrete piece or the core button and pusher may be integrally formed. In any case, pusher 340 moves forward relative to the implant (with a proximal end of the pusher separating and forming (and forming a gap, not shown) adjacent the implant capture feature shown as a form-fitting grip 396 included as part of the cap piece 390. This action turns the anchoring foot as desired (e.g., as shown in FIG. 19C).

Stated otherwise, the “softer” spring mechanically first bottoms out, which allows the center button to advance further upon continued application of force. Yet, while these actions are discussed as staged events, some relative movement of the center button 388 and pusher 340 occurs when advancing cap 30 (or withdrawing the cover and associated sheath) because of the relative spring rates. Nevertheless, the dual spring approach (with the optionally concentric parts pictured) provides for staged actuation of the sheath and pusher with a single user input motion.

It is also to be understood that the reversal of such action will occur upon button 388 release, thereby completing the anchoring foot deployment action (e.g., as show in FIG. 19D). Finally, the proximal end of the implant 110 may be released by opening the handle along its separation line 398 (shown in FIG. 22). This exposes half of the form-fitting grip 396 (referenced above) for the spring member body section 112.

Further note that the sheath 330 may comprise polyester (PET), PEEK or another high-strength material so that its wall thickness can be minimized. However, nylon (e.g., PEBAX) or another biocompatible material may be employed as may stainless steel hypotube material. Any other conventional material may be used for this and the other parts of the delivery system as well.

### Variations

Where a range of values is provided, it is understood that every intervening value, between the upper and lower limit of that range and any other stated or intervening value in the stated range is encompassed within the invention. Also, it is contemplated that any optional feature of the inventive variations described may be set forth and claimed independently, or in combination with any one or more of the features described herein. Moreover, no limitations from the specification are intended to be read into any claims, unless those limitations are expressly included in the claims.

As used herein and in the appended claims, the singular forms “a”, “an”, and “the” include plural referents unless the context clearly dictates otherwise. In other words, use of the articles allow for “at least one” of the subject items in the description above as well as the claims below. The claims may exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element irrespective of whether a given number of elements are enumerated in the claim, or the addition of a feature could be regarded as transforming the nature of an element set forth in the claims.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

The subject matter described herein and in the accompanying figures is done so with sufficient detail and clarity to permit the inclusion of claims, at any time, in means-plus-function format pursuant to 35 U.S.C. Section 112, Part (f). However, a claim is to be interpreted as invoking this means-plus-function format only if the phrase “means for” is explicitly recited in that claim.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, acts, steps, or elements of the embodiments may be recited in or added to the claims, as well as negative limitations that define the inventive scope of the claims by features, functions, acts, steps, or elements that are not within that scope.

1. A medical device comprising:
an implant comprising an elongate body and an oblong anchor rotatably attached at a distal end of the elongate body; and
a delivery system comprising a sheath for covering the elongate body and at least a portion of the anchor, a pusher received within a proximal end of the sheath;
wherein the pusher has an angled distal end configured to rotate the oblong anchor upon contact therewith when the anchor is free of the sheath.

2. The medical device of claim 1, comprising a dual spring rate actuator.

3. The medical device of claim 2, wherein a first spring provided in the actuator has a first spring rate, and second spring in the actuator has a higher second spring rate, thereby providing for staged actuation of the sheath and pusher with a single user input motion.

4. The medical device of claim 3, wherein the first and second springs are concentrically arranged.

5. The medical device of claim 1, comprising a releasable handle.

6. The medical device of claim 5, wherein the releasable handle is loaded with at least one anchoring head configured for securing position of the elongate body at a position opposite the oblong anchor serving as an anchoring foot.

7. A loader for applying an anchor over an elongate member, the loader comprising:
a body including a channel or tunnel portion configured to receive the elongate member; and
a plurality of flexible extensions from the body, each of the flexible extensions including an overhanding tip configured to retain the anchor.

8. The loader of claim 7, wherein an interior surface of each tip is ramped to permit anchor release when the anchor and the loader are pulled apart.

9. The loader of claim 7, further comprising the anchor and wherein a socket adjacent the extensions only permits receipt of the anchor within the tips when the anchor is inserted in the loader in the permitted direction.

10. A medical device comprising:
an elongate spring member comprising a plurality of cells including deflectable lateral beams connected at an outer extent and an extension section;
an oblong anchor rotatably connected at a distal end of the spring member extension section, and
an introduction needle extending past the oblong anchor.

11. The medical device of claim 10, wherein the needle is formed integrally with the spring member extension section.

12. The medical device of claim 11, wherein a waist section connects the needle to the spring member extension section and the waist section is formed perpendicular to the deflectable beams.

13. The medical device of claim 10, wherein the needle is tied by at least one strand to the spring member extension section through an eyelet.

14. The medical device of claim 10, wherein the needle is formed integrally with the oblong anchor.

15. A medical device comprising:
an elongate implant configured as a spring member comprising a section having a plurality of cells including deflectable lateral beams connected at an outer extent, and
a needle including at least one detent feature for spring member engagement and disengagement, engaged with the spring member.

16. A medical device comprising:
an elongate spring member comprising a section including a plurality of cells including deflectable lateral beams connected at an outer extent;
a screw head including a bone screw section and a proximal machine-screw section; and
a socket, the socket configured to receive the spring member at a proximal end and the machine-screw interface of the screw head at a distal end.

17. The medical device of claim 16, wherein the machine screw section includes at least one flat portion to provide a driver interface for the screw head.

18. The medical device of claim 1, wherein a distal end of the elongate spring member includes a tab having and eyelet, and a pin is received by the socket and through the eyelet.

19. The medical device of claim 18, wherein a portion of the tab is located outside the socket to provide a driver interface for the spring member and socket.

20. The medical device of claim 1, further comprising a proximal anchor engaged with the spring member.

21-30. (canceled)
